(19) United States
(12) Patent Application Publication
(10) Pub. No.: US 2026/0221229 A1
(43) Pub. Date: Jul. 30, 2026

(54) METHOD FOR SCREENING CROSS-SPECIES HGT

(71) Applicant: Yellow Sea Fisheries Research Institute, Chinese Academy of Fishery Sciences, Qingdao City (CN)

(72) Inventors: Naihao Ye, Qingdao City (CN); Xiao Fan, Qingdao City (CN); Yukun Wu, Qingdao City (CN); Xiaowen Zhang, Qingdao City (CN); Dong Xu, Qingdao City (CN); Yitao Wang, Qingdao City (CN); Ke Sun, Qingdao City (CN); Pengyan Zhang, Qingdao City (CN); Lepu Wang, Qingdao City (CN); Wei Wang, Qingdao City (CN)

(21) Appl. No.: 19/452,186

(22) Filed: Jan. 16, 2026

(30) Foreign Application Priority Data
Jan. 22, 2025 (CN) 202510103119.X

(51) Int. Cl. G16B 30/10; G16B 10/00; G16B 40/30

(52) U.S. Cl. CPC G16B 30/10; G16B 10/00; G16B 40/30

(57) ABSTRACT

A method for screening cross-species horizontal gene transfer (HGT). The method comprises steps of constructing a background database, inferring orthologous groups, constructing a gene phylogenetic tree, gene tree-based HGT inference, validation of inferred candidate HGTs based on a gene tree, and assigning HGTs to a timeline. By integrating multi-step processes such as phylogenetic analysis, sequence alignment, functional annotation, and statistical validation, the method achieves high-throughput and automated screening of HGT events in a large-scale gene data set. The method of the present invention is particularly suitable for HGT research across biological kingdoms with ultra-long time scales and ultra-long evolutionary distances, and is capable of providing an important scientific tool for understanding gene flow and functional enhancement during biological evolutionary history.

### TECHNICAL FIELD

The present invention belongs to the technical field of bioinformatics, and specifically relates to a method for screening cross-species HGT.

### BACKGROUND

Horizontal gene transfer (HGT), also referred to as lateral gene transfer (LGT), refers to a phenomenon where genetic material is exchanged between two species in a non-vertical inheritance relationship. This process is common in prokaryotes and is achieved through mechanisms such as conjugation, transduction, and transformation. Unlike vertical inheritance, the genetic mode from parents to offspring, HGT transcends the boundaries of kinship and greatly enriches the dynamics of gene flow.

The observation history of HGT dates back to 1959, when it was documented that high-frequency recombinant (Hfr) Escherichia coli could laterally transfer genetic information to specific mutants of Salmonella typhimurium. In the same year, Tomochiro Akiba and Kunitaro Ochiai discovered resistance plasmids in pathogenic bacteria and subsequently confirmed that these plasmids could transfer between different bacterial strains. However, the concept of HGT had not yet been formed at that time. It was not until the 1990s, with the emergence of genetically engineered organisms (GEOs), especially genetically engineered microorganisms (GEMs), and the appearance of numerous drug-resistant pathogens whose origins could not be solely attributed to gene mutations, that the concept of HGT gradually gained attention and became a research hotspot.

Observing similar phenotypes in organisms that are genetically distantly related is often attributed to shared genes in their genomes. These genes, which are absent in closely related lineages, may result from multiple independent gene loss events or may originate from HGT between different lineages. In theory, HGT can occur between any two organisms possessing DNA genomes. Unlike vertical gene transfer, which serves as the cornerstone for the preservation of biological heritage and the stability of the eukaryotic tree of life, HGT is an important force driving the diversification of eukaryotic species and assisting them in adapting to diverse environments. Although HGT has been widely recognized as a major evolutionary driver in prokaryotes, its role in eukaryotic evolution remains controversial, mainly due to complex evolutionary histories, complex genomic structures, and frequent sequence contamination issues.

To precisely identify HGT events, there is an urgent need to develop efficient and accurate screening methods. Currently, common HGT analysis methods cover phylogenetic tree analysis, base composition analysis, selection pressure analysis, intron analysis, specific sequence analysis, and nucleotide composition bias analysis. However, existing HGT screening methods mostly rely on phylogenetic analysis and have limitations such as incomplete database coverage, immature algorithms for automated tree selection, and a lack of unified analysis standards, which urgently require improvement and innovation.

### SUMMARY

The purpose of the present invention is to compensate for the deficiencies of the prior art and to provide an efficient and accurate method for screening cross-species HGT.

To achieve the above purpose, the technical solution adopted by the present invention is a method for screening cross-species HGT, comprising the following steps:
Step 1: constructing a background database comprising known protein sequences covering phylogenetic lineages of prokaryotes and eukaryotes, and removing highly homologous and redundant sequences;
Step 2: performing BLAST alignment on any two protein sequences in the background database, calculating an orthologous group association network based on reciprocal best hits for each gene pair combination, and clustering the orthologous group association network by using an unsupervised Markov clustering algorithm (MCL for short) to generate a database containing a plurality of orthologous groups;
Step 3: for each query protein sequence to be analyzed, searching for homologous sequences in the background database, performing rigorous sequence alignment and trimming, constructing a precise single-gene phylogenetic tree, and evaluating a support value of each branch of the phylogenetic tree through at least one of an ultrafast bootstrap test, an SH-like approximate likelihood ratio test, an approximate Bayes test, and a fast local bootstrap probability test;
Step 4: retrieving a nested topology of the query protein sequence and sequences that are distantly related in terms of evolutionary distance in the single-gene phylogenetic tree constructed centered on the query protein sequence; screening for a potential cross-species HGT by defining a nested position and setting a minimum support node threshold; and recording monophyletic best matching sequences of the query protein sequence in a recipient group and a donor group;
Step 5: evaluating confidence value of the potential cross-species HGT by calculating an alien index, an HGT score support index, an HGT branch length support index, and a consensus hit support index thereof;
Step 6: estimating a time of occurrence of a cross-species horizontal transfer event by tracing a minimum taxonomic boundary of descendants of the cross-species HGT and a minimum taxonomic boundary of descendants of the donor; and providing a temporal context for the cross-species HGT in combination with molecular clock dating and archaeological or fossil evidence.

Preferably, in the step 1, data of the background database comprise all available protein sequences of reference sequences in NCBI and protein sequences from a marine microbial eukaryote transcriptome sequencing project.

Preferably, in the step 2, multiple sequence alignment and protein domain analysis are performed on sequences in each of the orthologous groups.

Preferably, the step 5 further comprises analyzing the potential cross-species HGT using homology alignment characteristics of genes on both upper and lower sides of an HGT gene locus, specifically comprising:
if the potential cross-species HGT is located in a chromosomal fragment, and 50% of genes in the chromosomal fragment have best matches from other biological kingdoms, then removing the candidate gene of the potential cross-species HGT; or,
if the potential cross-species HGT is located in a chromosomal fragment, and 50% of genes in the chromosomal fragment are identified as HGT genes, then removing the candidate gene of the potential cross-species HGT; or,
if at least one of three nearest upstream or downstream genes of the potential cross-species HGT has a best hit in another biological kingdom, then excluding the potential cross-species HGT; or,
when two or more potential cross-species HGTs are physically tightly linked and belong to a same gene family, they are regarded as a single cross-species HGT event, and all these adjacent potential cross-species HGTs are retained.

Compared with the prior art, the method of the present invention has the following beneficial effects:
(1) High efficiency: through automated and standardized processes, high-throughput screening of HGT events in a large-scale gene data set is achieved, which greatly improves analysis efficiency.
(2) Accuracy: through multi-step processes such as constructing a large-scale background database, inferring orthologous groups, and constructing a gene phylogenetic tree, the accuracy of HGT screening is ensured. Meanwhile, by introducing multiple validation indices and taxonomic analysis of physical flanking genes, the false positive rate is further reduced.
(3) Wide applicability: the method is particularly suitable for HGT research of complex biological communities such as the kingdom Plantae, and is capable of providing an important tool for understanding gene flow and functional enhancement during biological evolutionary history.

### BRIEF DESCRIPTION OF THE DRAWINGS

FIG. 1 is a schematic diagram showing a system architecture and module composition used in a method for screening cross-species HGT in an embodiment of the present invention;

FIG. 2 is a schematic flowchart of a method for screening cross-species HGT in an embodiment of the present invention;

FIG. 3 shows a topology of evolutionary nodes of 113 organisms and the number of HGTs obtained at each node;

FIG. 4 shows evolutionary trends of codon usage bias over a timeline for HGTs of prokaryotic (Pr) and eukaryotic (Eu) origins from 113 organisms; a codon usage bias index of each species is normalized relative to its respective core genes, and the x-axis represents a chronological order in which HGTs were integrated into plant lineages; wherein, (a) and (b) are amino acid lengths of HGTs of prokaryotic (Pr) and eukaryotic (Eu) origins, respectively; (c) and (d) are amino acid aromaticity of HGTs of prokaryotic (Pr) and eukaryotic (Eu) origins, respectively; (e) and (f) are GC content at the third codon position of HGTs of prokaryotic (Pr) and eukaryotic (Eu) origins, respectively; (g) and (h) are codon adaptation indices of HGTs of prokaryotic (Pr) and eukaryotic (Eu) origins, respectively; (i) and (j) are frequency of optimal codons of HGTs of prokaryotic (Pr) and eukaryotic (Eu) origins, respectively; (m) and (n) are effective number of codons of HGTs of prokaryotic (Pr) and eukaryotic (Eu) origins, respectively;

FIG. 5 shows a comparison of codon usage bias between core genes (CORE) and horizontal gene transfer (HGT); wherein, (a) and (b) are effective number of codons (Nc) of HGTs of prokaryotic (Pr) and eukaryotic (Eu) origins in organisms of Rhodophyta, respectively; (c) and (d) are codon bias indices (CBI) of HGTs of prokaryotic (Pr) and eukaryotic (Eu) origins in organisms of Rhodophyta, respectively; (e) and (f) are effective number of codons (Nc) of HGTs of prokaryotic (Pr) and eukaryotic (Eu) origins in organisms of Chlorophyta, respectively; (g) and (h) are codon bias indices (CBI) of HGTs of prokaryotic (Pr) and eukaryotic (Eu) origins in organisms of Chlorophyta, respectively; (i) and (j) are effective number of codons (Nc) of HGTs of prokaryotic (Pr) and eukaryotic (Eu) origins in organisms of Streptophyta, respectively; (k) and (m) are codon bias indices (CBI) of HGTs of prokaryotic (Pr) and eukaryotic (Eu) origins in organisms of Streptophyta, respectively.

### DESCRIPTION OF THE EMBODIMENTS

The present invention will be further described below in conjunction with specific embodiments and the accompanying drawings. More details are set forth in the following description to facilitate a full understanding of the present invention. However, it is apparent that the present invention can be implemented in many other ways different from those described herein. Any alternative improvements or transformations made by those skilled in the art to the embodiments of the present invention shall fall within the protection scope of the present invention, and the protection scope of the present invention should not be limited by the content of these specific embodiments.

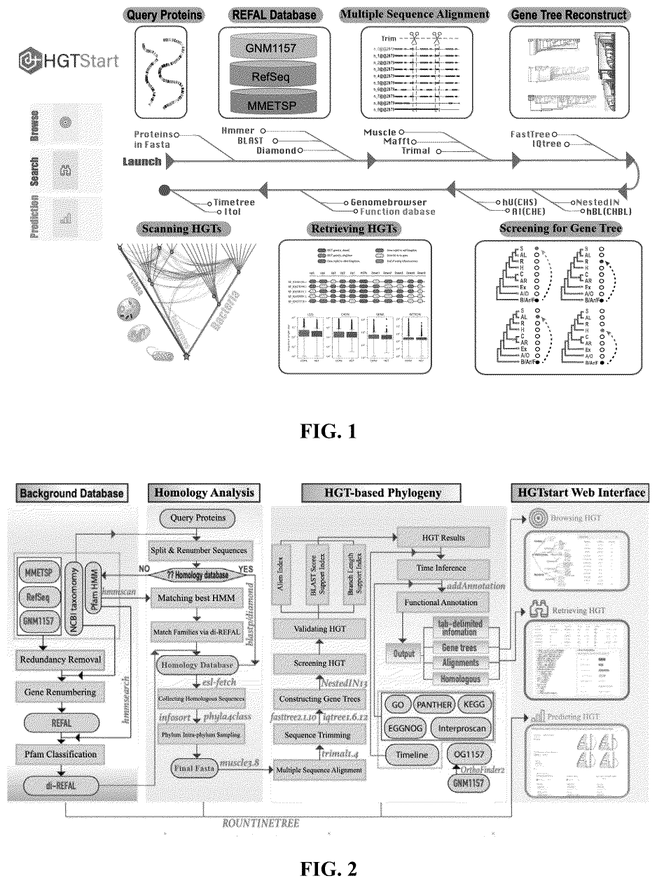

The present invention provides a method for screening cross-species HGT to facilitate large-scale, high-throughput HGT detection and to explore the breadth of HGT-driven biological evolution in the tree of life (TOL). The present invention applies the method to all species to generate a candidate HGT gene list and reveals distribution patterns of intra-kingdom and inter-kingdom HGTs. By arranging HGTs in chronological order, their contributions to the complexity and diversification of biological systems are inferred, providing the possibility to validate numerous evolutionary theories. The system (HGTStart) used in the method is shown in FIG. 1, and the process is shown in FIG. 2, comprising the following steps:

### 1. Construction of a Background Database

In order to conduct an effective HGT investigation, the present invention includes as many high-quality protein sequences as possible from whole-genome data of a wide range of prokaryotic and eukaryotic lineages, as well as individual sequences from major protein databases.

The present invention examined the genome assembly summary information of RefSeq (ftp://ftp.ncbi.nlm.nih.gov/genomes/README_assembly_summary.txt, updated to May 2021) to download all available proteins in genomes completed before the update date. The present invention grouped genomes of species within the same genus, wherein only the genome representing the least fragmented assembly in the group was downloaded (if there were multiple versions of the genome, the latest version was selected). The present invention also searched for genomes from JGI (https://genome.jgi.doe.gov/portal/) and other databases.

An important issue affecting HGT inference is uneven sampling caused by imbalanced data collection among different taxa. The present invention incorporated proteins from MMETSP into the database to compensate for the small amount of genomic data of Rhodophyta. These extensive genomic data formed a protein database containing 17,250,679 protein sequences from 1,157 genomes, which have reasonable coverage in most lineages in the tree of life (including 540 bacterial, 45 archaeal, 431 opisthokont, 15 rhodophyte, 83 chlorophyte, and 43 genomes from lineages of Chromalveolata). Each of the 1,157 complete genomes represents a representative species of its genus. The database was named “GNM1157”.

In addition to GNM1157, the present invention also constructed a larger database containing as many known protein sequences as possible. The present invention constructed this background database by downloading the NCBI RefSeq database (Version 82, ftp://ftp.ncbi.nlm.nih.gov/refseq/). Given the underrepresentation of algal lineages in RefSeq, the present invention included abundant algal protein data from the MMETSP project and other public sources to expand the taxonomic span of the background database.

The collected protein sequences (including GNM1157, RefSeq, and MMETSP data) were integrated into a main database REFAL, and highly similar sequences (sequence identity ≥90%) in each taxon (such as Brassicaceae or Primates) were removed using CD-HIT version 4.5.4. Finally, a protein database containing 39.9 million sequences from over 7,786 taxa was constructed, ensuring reasonable coverage in most lineages of the tree of life.

To facilitate functional prediction of a query sequence, the present invention predicted protein sequences of GNM1157 by accessing large-scale databases such as InterProScan, eggNOG, PANTHER, Pfam, and SUPERFAMILY. The query sequence is associated with its gene hit in GNM1157 to retrieve the corresponding predicted function.

### 2. Orthologous Group Inference and Benchmarking

Homology relations between genes, including orthology and paralogy, are the cornerstone of comparative biological research (such as horizontal gene transfer analysis). Currently, tree-based databases such as PhylomeDB, Ensembl-Compara, eggNOG, and TreeFam have been developed in this field. However, the present invention found that these existing databases failed to comprehensively cover the entire gene set encoded in GNM1157; therefore, constructing a customized orthology database is particularly important. The present invention clustered 17,250,679 proteins encoded in GNM1157 and integrated protein-based topology analysis. All protein sequences were exhaustively analyzed through an all-against-all BLAST workflow of whole genomes (Version 2.2.28, with an e-value threshold set to 1e-10 and a local identity threshold set to 20%). Subsequently, using OrthoFinder V2.3.7 software, an orthologous group association network was calculated based on reciprocal best length-normalized hit (RBNH) information for each genome pair combination. OrthoFinder excels in correcting the dependence of gene similarity on gene length and phylogenetic distance, thereby improving the accuracy of orthologous group definition. In addition, the present invention independently ran an unsupervised Markov clustering algorithm (MCL 3.0) to cluster this orthologous group graph. By adopting a set of gradient inflation parameters (1.2, 1.4, 1.6, 1.8, 2.0, 2.2), the present invention ran the MCL algorithm multiple times and selected the best-performing result. The present invention locally downloaded more than ten most popular online orthology databases (for example, eggNOG, PANTHER, and SUPERFAMILY) as benchmarks to calibrate orthology inference parameters of the present invention. Precision, recall, and F0 values of the gradient set were calculated using the NumPy library in Python 3.0 to determine an optimal value for the inflation coefficient.

Wherein, TP is the number of true positive orthologous group assignments (i.e., correct assignments), FP is the number of false positive orthologous group assignments (i.e., incorrect assignments), and FN is the number of false negative orthologous group assignments (i.e., missed assignments). The F-score is the harmonic mean of precision and recall, which is capable of balancing these two indices and is more sensitive to lower indices, facilitating a comprehensive evaluation of orthologous group assignment performance.

In order to identify biologically meaningful orthologous clusters, the present invention tested group-specific conserved domain performance for sequences in each orthologous group (OG), and filtered out noise and weakly associated sequences. First, multiple sequence alignment was performed for sequences within each OG using MAFFT v7.455. Next, an HMM configuration file of a conserved domain was constructed for the alignment results of each OG using HMMER 3.0. Subsequently, the present invention searched all protein sequences in each OG against their corresponding HMM configuration files using the Hmmsearch program with an E-value threshold of 0.00001. Sequences below the threshold were removed from the group, and these sequences were collected for a second round of sequence orthology construction. After this round of screening, each sequence was accurately assigned to an orthologous group. Finally, this clustering process yielded 27,631 orthologous groups, with each group containing at least two members. For orthologous groups with five or more members, the present invention further examined their phylogenetic relationships by constructing maximum likelihood trees. Specifically, sequences were aligned using MUSCLE version 3.8.31 with default settings, and then alignment results were trimmed using trimAl version 1.232 in an automatic mode (-automated1), retaining an alignment length of at least 50 amino acids.

These trimmed alignment results (≥50 amino acids) were used to construct phylogenetic trees under FastTree version 2.1.7 using the “WAG+CAT” model. During the construction process, four rounds of minimum-evolution SPR (subtree pruning and regrafting) moves (-spr 4) and exhaustive maximum likelihood nearest-neighbor interchanges (-mlacc 2 -slownni) were performed. Branch support values were estimated through the Shimodaira-Hasegawa (SH) test.

Phylogenetic trees successfully reconstructed using all members in the OG indicate that the orthologous group clustering strategy based on sequence similarity is strongly supported by the phylogenetic strategy. These trees are capable of clearly distinguishing nested positions formed by taxonomically distantly related sequences at variable evolutionary rates, thereby elucidating gene relationships of horizontal gene transfer (HGT).

### 3. Construction of a Gene Phylogenetic Tree

The present invention provides a “RoutineTree” model for unsupervised homology search, sequence alignment, tree construction, and tree screening to predict HGT for each given protein sequence. Briefly, RoutineTree searches for homologous sequences in REFAL and constructs a single-gene phylogenetic tree for each customized query protein sequence. In order to speed up the search process, an HMM model and a probabilistic inference method integrated in HMMER 3.0 are used to construct database segments based on functional conserved domains. By using hmmscan to scan 159,424 configuration files from the PFAM database, the REFAL database is divided into families, and then sequences are retrieved using esl-fetch. Each family is named after its corresponding PFAM number for subsequent retrieval. Excluded REFAL sequences are collected into a FASTA file.

At the beginning, a query sequence is split into individual sequence files in the first step, and each is scanned against PFAM HMM configuration files using hmmscan. A temporary BLAST/Diamond database is constructed based on a hit PFAM number of each split query sequence, from which corresponding FASTA files in REFAL are retrieved and merged. After making a retrieval index in BLAST/Diamond format, the temporary database is searched using a default e-value threshold which is equal to 1e-05. For each query, the top 10,000 significant hits sorted by bit score in descending order (by default) are recorded. Sequences corresponding to the hits are retrieved from the temporary database, with no more than three sequences per genus and no more than ten sequences per phylum. Significant hits with a query-hit alignment length of at least 120 amino acids are subsequently re-sorted in descending order of query-hit identity. Homologous sequences plus the query are combined and aligned using MUSCLE version 3.8.31 under default settings. The resulting alignment is trimmed using TrimAl version 1.2 in an automatic mode (-automated1). The present invention discards queries with amino acid compositions significantly different (P<0.05) from the remaining sequences in the alignment. These trimmed alignments (≥50 amino acids) are used to construct phylogenetic trees under IQtree multi-core version 1.6.12 using an optimal model calculated by ModelFinder in IQtree. Branch support values are estimated through an ultrafast bootstrap (UFboot, -bb 1500) test, a Shimodaira-Hasegawa-like approximate likelihood ratio test (SH-aLR T, -alrt 1200), an approximate Bayes test (-abayes), and a fast local bootstrap probability test (-bb 1500).

### 4. Tree-Based HGT Inference

In a phylogenetic tree, the present invention is dedicated to searching for a heterologous nested topology of a query protein sequence. To this end, the present invention integrates NestedIn, which is a phylogenetic tree scanning tool implemented in Java, into the RoutineTree model to screen for heterologous nested positions existing in a gene tree. The so-called heterologous nested position refers to two or more monophyletic groups in the tree containing the query protein sequence and sequences that are distantly related in terms of genetic distance, and these monophyletic groups are supported by different nodes. This essentially reflects a conflict between the gene tree and a species tree. In order to enhance the applicability and flexibility of the tool, NestedIn specifically provides a user-friendly parameter “-donor”, allowing a user to input any taxonomic node for which a horizontal gene transfer (HGT) relationship with the query protein sequence is to be tested.

On the query (recipient) side, in order to ensure that all descendant genes in a monophyletic group after the occurrence of HGT are included, the NestedIn tool provides an “-optional” parameter, allowing the user to input an ancestral level of the query protein sequence. Specifically, the workflow of NestedIn is as follows:

First, it reads a tree file in Newick format and parses the topology of the tree. Next, starting from the query protein sequence, it traverses its superior nodes step by step. During this process, NestedIn records each node matching a nested criterion set by the user until it reaches a first leaf node that is neither a donor nor optional. There may be multiple nodes meeting filtering criteria. The determination of a final node follows the following principles: 1) in order to exclude interference from contamination and recent HGT events as much as possible, NestedIn removes singletons in donor and recipient genes and allows the user to customize a minimum number requirement; 2) only nested positions that are multi-supported by a customized threshold (default settings: SH test ≥0.70 and aBayes test ≥0.70) in support nodes are retained; 3) among all remaining nodes, a node with a highest taxonomic level is selected as a final candidate HGT node.

For a given nested position, NestedIn provides monophyletic best matching sequences (MMSHs) of the query protein sequence in a recipient group (MMSH_IN) and a donor group (MMSH_OUT). This is done to obtain predicted functional information (MMSH_IN) and to list a representative donor gene (MMSH_OUT) for analyzing evolutionary diversification between the recipient and the donor after the occurrence of HGT.

### 5. Validation of Tree-Inferred HGT Candidates

In order to construct a systematic method to quickly, comprehensively, and reliably detect HGT, the present invention utilizes an alien index (AI), an HGT score support index (hU), an HGT branch length support index (hBL), and a consensus hit support index thereof to test confidence value of HGT candidates.

First, the present invention defines several concepts for a query protein sequence in a monophyletic group in a gene tree:
INGROUP: all sequences containing the query protein sequence within a level of a grade specified by the user.
OUTGROUP: all sequences outside the level of the grade defined by the user (i.e., sequences not belonging to the INGROUP).
SKIPGROUP: the query protein sequence itself and sequences at a lower grade level specified by the user at the INGROUP boundary; sequences in the SKIPGROUP are assumed to be homologous sequences that may originate after an HGT event.

### (1) Alien Index (AI)

The present invention calculates an AI score for each query gene using the e-value of the BLAST index:

AI=(bbhO/bbhG)

Wherein, bbhG is the e-value of a best hit in an INGROUP lineage, and bbhO is the e-value of a best BLAST hit in an OUTGROUP lineage. E-values in the SKIPGROUP are skipped because they are assumed to be homologous sequences after HGT. When no significant BLAST hit is detected, the corresponding bbhG or bbhO is set to 1. The AI score serves as an indicator of a degree of similarity of the query protein sequence to its homologous sequences in the INGROUP compared to its homologous sequences in the OUTGROUP. Since all BLASTp searches are performed using the same database, it is reasonable to apply a uniform threshold to all query taxa.

In a preliminary screening stage, the present invention selects a relatively loose AI score threshold (AI>0). In a subsequent screening stage, this threshold can be adjusted according to specific analysis needs. As a practical choice, in the research of the present invention, a moderately less strict threshold (AI>10) compared to previous studies was chosen because it yielded desired results.

### (2) HGT Score Support Index (hU)

The present invention calculates a hU score for each query gene based on best alignment scores of the INGROUP and the OUTGROUP:

hU=(Best hit bit score of OUTGROUP)−(Best hit bit score of INGROUP)

Bit scores in the SKIPGROUP are skipped because they are assumed to be homologous sequences after HGT. When no significant BLAST hit is detected, the best hit bit scores of the OUTGROUP and the INGROUP are set to 0, respectively. The hU score reflects a degree of similarity of the query to its homologous sequences in the INGROUP compared to its homologous sequences in the OUTGROUP. Since all BLASTp searches are performed using the same REFAL database, it is reasonable to apply a uniform threshold to all query taxa. Considering its combination with other criteria in HGT inference, in the preliminary screening stage, the present invention selects a relatively loose hU score threshold (hU>0).

(3) HGT Branch Length Support Index (hBL)

The present invention develops a new index, hBL (HGT Branch Length Support Index), for each query gene, which is based on a minimum branch length from a query to the INGROUP and the OUTGROUP:

hBL=(minimum branch length from the INGROUP to the query)−(minimum branch length from the OUTGROUP to the query).

Branch length values in the SKIPGROUP are skipped because they are assumed to be homologous sequences after HGT. When no gene is detected, the minimum branch lengths from the INGROUP and the OUTGROUP to the query are set to 100, respectively. The hBL score serves as an indicator of a degree of similarity of the query to its homologous sequences in the INGROUP compared to its homologous sequences in the OUTGROUP. For each leaf node in a tree, a branch length to the query is determined by summing all branch lengths connecting the leaf node to the query. Since all branch lengths are specific to the same tree, a direct comparison between the INGROUP and the OUTGROUP can be performed by summing these lengths. In a preliminary screening stage, the present invention applies a relatively loose hBL score threshold (hBL>0), considering its combination with other criteria in HGT inference. In a subsequent screening stage, a user retains flexibility to adjust this threshold according to specific analysis needs.

### (4) Consensus Hit Support

Considering a possibility of accidental introduction of sequence contamination into the INGROUP or the OUTGROUP, the present invention calculates consensus hit support for the AI, the hU, and the hBL. The consensus hit support measures a degree of support provided by all genes in the OUTGROUP (rather than just a best-hit gene). The specific indices are as follows:

Consensus Hit Support-E-value (CHE): This index represents a ratio of a number of genes in the OUTGROUP with an E-value less than bbhG (the best hit in the INGROUP) to a total number of genes in the OUTGROUP. The CHE serves as a confidence indicator for the condition AI>0.

Consensus Hit Support-Score (CHS): The CHS represents a ratio of a number of genes in the OUTGROUP with a score greater than bbhG to the total number of genes in the OUTGROUP. This index serves as a confidence indicator for the condition hU>0.

Consensus Hit Support-Branch Length (CHBL): The CHBL is calculated as a ratio of a number of genes in the OUTGROUP with a branch length less than bbhG to the total number of genes in the OUTGROUP. It serves as a confidence indicator for the condition hBL>0.

These consensuses hit support indices provide additional insights into the reliability of the AI, hU, and hBL conditions by considering a broader range of genes in the OUTGROUP.

In addition to the above strategies, the present invention further implements the following criteria to evaluate taxonomy of physical flanking genes of a potential candidate HGT to eliminate a possibility of contamination:
(1) If an HGT candidate gene is located in a contig (referring to a set of overlapping DNA segments) wherein best hits of 50% of genes are in other kingdoms, the candidate gene is excluded.
(2) If the HGT candidate gene is located in a contig wherein 50% of genes are primarily identified as HGT genes, it is excluded.
(3) If at least one of three nearest flanking genes (upstream and downstream) of the HGT candidate gene has a best hit in another kingdom, it is excluded.
(4) When two or more HGT genes are physically tightly linked and belong to a same gene family, they are regarded as a single HGT event, and all these HGTs are retained for further analysis.

### 6. Assigning HGT to a Timeline

In order to better understand impacts of HGT events on the evolutionary history and geological changes of the Earth and to place these events in a temporal context, the present invention aims to estimate occurrence times of these HGT events. However, tracing an exact time of gene divergence based on accumulation of nucleotide mutations becomes increasingly challenging over time. As time passes, a probability of multiple nucleotide substitutions at a single site increase, as does an occurrence of unexpected events such as gene loss and gene duplication. An objective of the present invention is to provide abroad overview of HGTs during the existence of organisms, rather than to delve into complexities of evolutionary algorithms.

Nevertheless, the present invention can establish general upper and lower limits for the timing of HGT events based on the following consensus principle: vertical inheritance is unidirectional. For example, if a successful prokaryote-to-eukaryote HGT event occurred at a specific evolutionary node, such as a direct ancestor of modern brown algae, which is a unicellular organism, then a homologous gene resulting from the HGT can spread to descendants of the brown algae node through vertical inheritance, but cannot spread to an ancestral node, the Stramenopiles.

For a given query protein sequence determined as a potential HGT, if the present invention can find all descendants derived from an initial HGT event, the present invention can trace back to a common ancestor node. An occurrence time of this common ancestor can be inferred through a molecular clock dating method combined with archaeological and fossil evidence. Technically, a minimum taxonomic boundary of HGT descendants can be traced using all gene members in the INGROUP, while a minimum taxonomic boundary of donor descendants can be traced using all gene members in the OUTGROUP.

Accurate taxonomy is crucial for representing a hierarchy of evolutionary nodes and organism relationships. To this end, the present invention uses the NCBI taxonomy system, which is used to classify all life forms on Earth due to its widely accepted and reasonable classification framework. The present invention manually introduced a kingdom-level node, Chromalveolata, into the NCBI taxonomy system, and proposed reclassifying Cryptophyta, Rhizaria, Apicomplexa, Stramenopiles, and Dinoflagellata into this kingdom based on recent research. Timeline data for intermediate nodes are primarily derived from the TimeTree database.

### 7. Output and Visualization

For each individual query protein, the present invention provides its homologous genes along with corresponding alignments and phylogenetic trees. For finally determined HGT genes, detailed information is provided in a tab-delimited text file, including a donor node and a recipient node and their occurrence times, MMSH genes in the INGROUP and the OUTGROUP, AI, hU, and hBL scores and support indices thereof, and predicted functional information from multiple databases. Accession numbers listed in the table are hyperlinked to corresponding external databases such as GO and KEGG. As shown in Table 1, output information for part of the query proteins is presented.

TABLE 1
Output information of query proteins

| | #GeneName | DonorNode | DNtime | ReceptorNode | RNtime | MSMH_OUT |
|---|---|---|---|---|---|---|
| 1 | Viridiplantae-- | Bacteria.Bacte | 3936 | Eukaryota. | NA | Bacteria.Chl |
| | | | | Archaepl | | |
| 2 | Viridiplantae-- | Eukaryota.Euka | 2101 | Eukaryota. | 117 | Chromalveola |
| | | | | Viridipl | | |
| 3 | Viridiplantae-- | Bacteria.Bacte | 3936 | Eukaryota. | 117 | Bacteria--Pr |
| | | | | Viridipl | | |
| 4 | Viridiplantae-- | Eukaryota.Meta | 180 | Eukaryota. | 117 | Opisthokonta |
| | | | | Viridipl | | |
| 5 | Viridiplantae-- | Bacteria.Bacte | 3936 | Eukaryota. | 160 | Bacteria--FC |
| | | | | Viridipl | | |
| 6 | Viridiplantae-- | Bacteria.Bacte | 3936 | Eukaryota. | 117 | Bacteria.Cya |
| | | | | Viridipl | | |
| 7 | Viridiplantae-- | Eukaryota.Euka | 2101 | Eukaryota. | 1160 | Chromalveola |
| | | | | Viridipl | | |
| 8 | Viridiplantae-- | Eukaryota.Chro | NA | Eukaryota. | 110 | Chromalveola |
| | | | | Viridipl | | |
| 9 | Viridiplantae-- | Eukaryota.Meta | 753 | Eukaryota. | 117 | Opisthokonta |
| | | | | Viridipl | | |
| 10 | Viridiplantae-- | Eukaryota.Viri | 431 | Eukaryota. | 96 | Plantae.Viri |
| | | | | Metazoa. | | |

| | MSMH_IN | AI (CHE) | hU(CHS) | hBL(CHBL) | GOTerm | KEGGTerm |
|---|---|---|---|---|---|---|
| 1 | Viridiplan | 32.61 (87.5) | 85.7 (87.5) | 1.67 (100.0) | GO:0001522\|G | NA |
| 2 | Viridiplan | 6.37 (100.0) | 55.5 (100.0) | 96.81 (100.0) | GO:0003674\|G | MetaCyc: |
| | | | | | | PWY-7511 |
| 3 | Viridiplan | 102.24 (100.0) | 319.0 (100.0) | 98.38 (00.0) | NA | NA |
| 4 | Plantae.Vi | 7.0 (100.0) | 58.5 (100.0) | 96.11 (100.0) | NA | NA |
| 5 | Viridiplan | 53.21 (100.0) | 181.0 (100.0) | 98.8 (100.0) | GO:0003674\|G | NA |
| 6 | Viridiplan | 18.44 (100.0) | 73.2 (100.0) | 98.55 (100.0) | GO:0000312\|G | Reactome: |
| | | | | | | R-HSA-53 |
| 7 | Plantae.Vi | 22.77 (100.0) | 85.1 (100.0) | 98.52 (100.0) | GO:0000993\|G | Reactome: |
| | | | | | | R-HSA-16 |
| 8 | Plantae.Vi | 19.9 (100.0) | 97.4 (100.0) | 98.11 (100.0) | GO:0003008\|G | NA |
| 9 | Viridiplan | 71.39 (100.0) | 230.0 (100.0) | 98.79 (100.0) | NA | NA |
| 10 | Opisthokon | 200.0 (100.0) | 1385.0 (100.0) | 100.0 (100.0) | GO:0000003\|G | NA |

The feasibility of the method of the present invention is verified using the following methods:

1. Determining Core Genes and Conducting In-Depth Comparison with HGT Genes

Core genes are typically present in genomes of almost all members of a species or taxon, and they constitute a most conserved gene set. Identifying these core genes and contrasting them with HGT is a fundamental pursuit of evolutionary genomics. This effort enables researchers to utilize host core genes as reference points to evaluate adaptation and evolution of HGT, revealing genetic mechanisms of adaptation and diversification in the domain of organisms. To achieve these goals, the present invention compares GC ratios, codon usage, and gene structures to find differences between HGT and CORE genes.

### (1) Determination of Core Genes

A basic criterion is that if a gene family is capable of containing at least 70% of species within a phylum, it is regarded as a core gene family of the phylum, and its members are designated as core genes.

### (2) Codon Usage

Codon usage is an important factor in determining a fate of HGT because it is compatible with gene transcription machinery and tRNA pools in a host. The present invention calculated codon usage and GC content indices using CodonW version 1.4.4 (http://codonw.sourceforge.net). A correlation test between CAI and gene expression was performed using a Spearman rank correlation analysis tool (P. Wessa, Free Statistics Software, Office of Research and Development Education, version 1.1.23-r7, https://www.wessa.net/).

The present invention first utilizes CodonW to analyze various gene parameters for each species (number of synonymous codons (L_sym), total number of amino acids (L_aa), G+C content (GC), G+C content at the third codon position (GC3s), A/T/C/G content at the third codon position (A3s, T3s, G3s, C3s)), codon usage indices (Codon Adaptation Index (CAI), frequency of optimal codons (Fop), effective number of codons (Nc), codon bias index (CBI)), and amino acid indices (hydrophilicity (GRAVY score), protein aromaticity (Aromo)). Codon usage bias for each gene of each species was analyzed. Then, the present invention identifies a most conserved gene in each species as a core gene of this species. For each species, the present invention classifies HGT genes based on transfer nodes (red plants, green algae, chlorophytes, streptophytes, embryophytes, tracheophytes, mesangiosperms, rosids, and plants) and donor categories (prokaryotes and eukaryotes).

To determine a testing method for the codon usage bias, tests for normality and homogeneity of variance were first performed on each codon bias index of core genes (CORE) and HGT genes for each species using SPSS. A K-S test was performed on 14 codon usage bias indicators (T3s, C3s, A3s, G3s, CAI, CBI, Fop, Nc, GC3s, GC, L_sym, L_aa, Gravy, and Aromo) of 113 species. If an asymptotic significance is >0.05, it represents that the indicator conforms to a normal distribution; otherwise, it does not. Taking 9 species such as cyanophaophora as examples, the codon usage bias indicators of each species are all less than 0.05, representing that none of them conform to the normal distribution. Since parametric tests require that data of each group must conform to the normal distribution, a non-parametric testing method needs to be used when testing for differences between the CORE genes and the HGT genes, as well as between transfer genes at adjacent nodes across different phyla. The K-S test was performed on the 14 codon usage bias indicators (T3s, C3s, A3s, G3s, CAI, CBI, Fop, Nc, GC3s, GC, L_sym, L_aa, Gravy, and Aromo) of the 113 species. If the asymptotic significance is >0.05, it represents that the indicator conforms to the normal distribution; otherwise, it does not. Taking the 9 species such as cyanophaophora as examples, the codon usage bias indicators of each species are all less than 0.05, representing that none of them conform to the normal distribution. Since parametric tests require that data of each group must conform to the normal distribution, the non-parametric testing method needs to be used when testing for differences between the CORE genes and the HGT genes, as well as between transfer genes at adjacent nodes across different phyla. The results are shown in Table 2 and Table 3.

TABLE 2
Statistical description

| | Number of Cases | Mean | Standard Deviation | Minimum | Maximum |
|---|---|---|---|---|---|
| T3s | 8319 | .083987 | .0627330 | .0000 | .3684 |
| C3s | 8319 | .571169 | .0817237 | .2568 | .8750 |
| A3s | 8319 | .066231 | .0527260 | .0000 | .3788 |
| G3s | 8319 | .482963 | .0697491 | .1562 | .7442 |
| CAI | 8319 | .23701 | .042212 | .068 | .502 |
| CBI | 8319 | .17591 | .071341 | −.206 | .574 |
| Fop | 8319 | .51669 | .042003 | .344 | .771 |
| Nc | 8319 | 36.2235 | 6.46109 | 23.57 | 61.00 |
| GC3s | 8319 | .87423 | .088762 | .395 | 1000 |
| GC | 8319 | .68557 | .062812 | .460 | .961 |
| L_sym | 8319 | 590.48 | 558.852 | 58 | 8251 |
| L_aa | 8319 | 607.81 | 572.798 | 59 | 8423 |
| Gravy | 8319 | −.26585281 | .345545987 | −2.194321 | 1.064762 |
| Aromo | 8319 | .06881480 | .025602160 | .000000 | .216783 |

a. Species = Cyanophaophora

TABLE 3
One-sample Kolmogorov-Smirnov test

| | | T3s | C3s | A3s | G3s | CAI | CBI | Fop |
|---|---|---|---|---|---|---|---|---|
| Number of | | 8319 | 8319 | 8319 | 8319 | 8319 | 8319 | 8319 |
| Cases | | | | | | | | |
| Normal | Mean | 0.083987 | 0.571169 | 0.066231 | 0.482963 | 0.23701 | 0.17591 | 0.51669 |
| Parametersb,c | | | | | | | | |
| | Standard | 0.062733 | 0.0817237 | 0.052726 | 0.0697491 | 0.042212 | 0.071341 | 0.042003 |
| | Deviation | | | | | | | |
| Most Extreme | Absolute | 0.148 | 0.053 | 0.144 | 0.019 | 0.059 | 0.038 | 0.03 |
| Differences | Positive | 0.148 | 0.029 | 0.144 | 0.019 | 0.059 | 0.03 | 0.03 |
| | Negative | −0.112 | −0.053 | −0.107 | −0.009 | −0.038 | −0.038 | −0.029 |
| Test Statistic | | 0.148 | 0.053 | 0.144 | 0.019 | 0.059 | 0.038 | 0.03 |
| Asymptotic | | .000d | .000d | .000d | .000d | .000d | .000d | .000d |
| Significance | | | | | | | | |
| (2-tailed) | | | | | | | | |

| | | Nc | GC3s | GC | L_sym | L_aa | Gravy | Aromo |
|---|---|---|---|---|---|---|---|---|
| Number of | | 8319 | 8319 | 8319 | 8319 | 8319 | 8319 | 8319 |
| Cases | | | | | | | | |
| Normal | Mean | 36.2235 | 0.87423 | 0.68557 | 590.48 | 607.81 | −0.26585281 | 0.0688148 |
| Parametersb,c | | | | | | | | |
| | Standard | 6.46109 | 0.088762 | 0.062812 | 558.852 | 572.798 | 0.345545987 | 0.2560216 |
| | Deviation | | | | | | | |
| Most Extreme | Absolute | 0.148 | 0.145 | 0.043 | 0.173 | 0.173 | 0.043 | 0.046 |
| Differences | Positive | 0.148 | 0.108 | 0.025 | 0.169 | 0.168 | 0.043 | 0.046 |
| | Negative | −0.106 | −0.145 | −0.043 | −0.173 | −0.173 | −0.041 | −0.029 |
| Test Statistic | | 0.148 | 0.145 | 0.043 | 0.173 | 0.173 | 0.043 | 0.046 |
| Asymptotic | | .000d | .000d | .000d | .000d | .000d | .000d | .000d |
| Significance | | | | | | | | |
| (2-tailed) | | | | | | | | |

a Species = Cyanophaophora
bTest distribution is normal
cCalculated from data
dLilliefors Significance Correction

Since the data for each species cannot simultaneously satisfy normality and homogeneity of variance, the present invention uses a Mann-Whitney U test in non-parametric tests to detect differences between the CORE genes and the HGT genes, as well as between transfer genes at adjacent nodes across different phyla. The results are shown in FIG. 5, wherein red dots represent the core genes, blue dots represent HGT of prokaryotic (Pr) origin, and green dots represent HGT of eukaryotic (Eu) origin. Each dot on a same horizontal coordinate represents one species. The horizontal coordinate represents, from left to right, the core genes and transfer nodes arranged chronologically from ancient to recent. A connecting line represents a difference between adjacent horizontal coordinates of the species. A red line represents that a codon bias index of an HGT at a recent node is significantly increased compared to an HGT at an ancient node of this species, a blue line represents a significant decrease, and a gray line represents no significant difference.

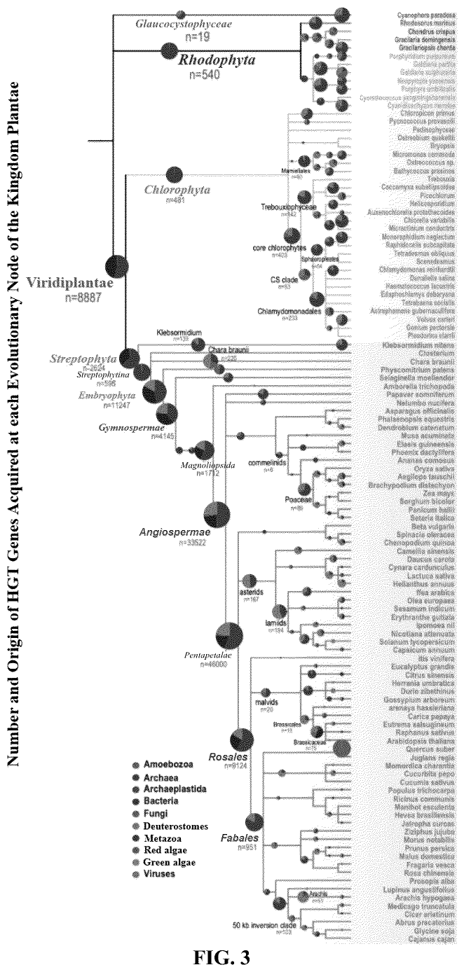

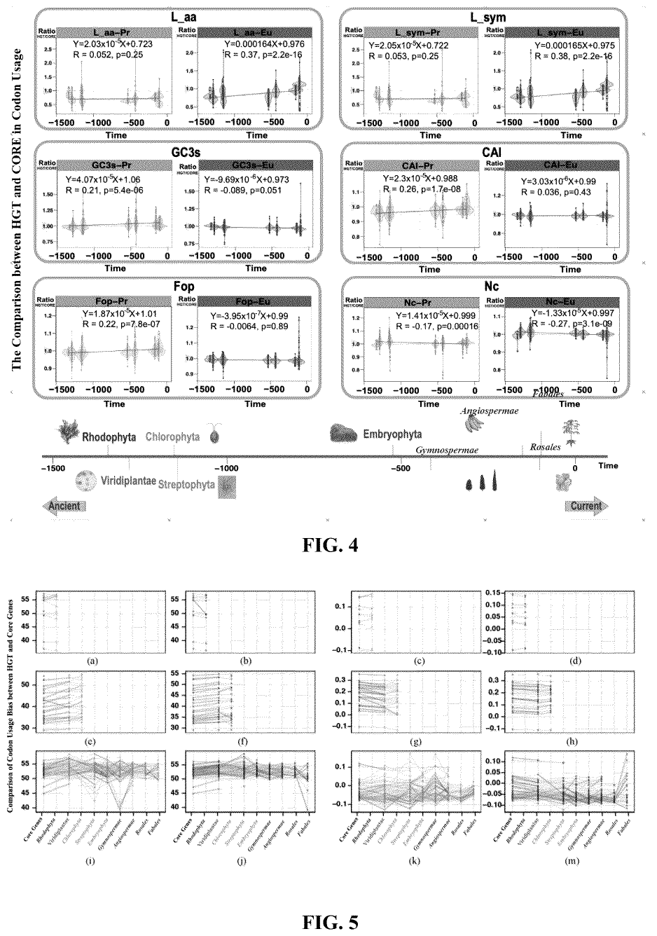

In order to better compare the HGT genes with the core genes, the present invention normalizes the HGT genes using the core genes, compares codon usage bias of all nodes using violin plots, and establishes a correlation between the codon usage bias and transfer time. Since an evolutionary rate of each species is different, the present invention uses the core genes of respective species as a reference standard for horizontal comparison. A codon bias index of the HGT of each species was normalized relative to the codon bias index of its respective core genes, i.e., by dividing the codon bias index of the HGT by the codon bias index of the core genes. The codon usage bias of different species at all nodes is presented using violin plots, and a correlation between the codon usage bias and the transfer time is established. FIG. 3 and FIG. 4 show temporal trends of codon bias of horizontal gene transfer (HGT). As shown in FIG. 4, each dot on a same horizontal coordinate represents a normalized codon usage bias index of a species. HGT genes obtained at different evolutionary nodes are compared according to their chronological order. Compared parameters include L_aa, L_sym, GC3s, CAI, Fop, and Nc, and detailed explanations of each parameter are as follows: before 1,000 MYA, genes transferred from eukaryotes and prokaryotes into plants have similar L_aa and L_sym, which are significantly shorter than those of the core genes, being about 75% of the core genes. It is widely reported that long fragments of exogenous DNA undergo faster inactivation and are less likely to be fixed in a genome of a recipient organism than short insertions. However, a significant difference is that L_aa and L_sym of genes transferred from eukaryotes to plants steadily increase over time, and in recent times, have become very close to those of the core genes. This difference may originate from a proximity between plants and donors. Obviously, compared to prokaryotes, plants are closer to other eukaryotes and have more similar genomic compositions, which is more conducive to fixation of long-fragment exogenous genes.

GC3s exhibits completely different trends in genes of eukaryotic and prokaryotic origins. The GC3s of genes of prokaryotic origin has always been higher than that of host core genes and becomes higher and higher over time. In contrast, the GC3s of genes of eukaryotic origin has always been lower than that of the host core genes and becomes lower and lower over time. This well demonstrates a process of “domestication,” i.e., genes acquired earlier evolve along with the host genome, and their codon usage becomes closer and closer to that of the core genes of the host itself.

CAI refers to a degree of consistency between usage frequencies of synonymous codons and optimal codons in a coding region. By comparing a codon usage frequency of a specific gene with a reference set of high-expression genes of a species, a CAI of the specific gene can be determined. A CAI score of a gene is calculated based on usage frequencies of all codons in this gene. CAI is often used to evaluate an expression level of an exogenous gene in a host; the higher the CAI, the higher the expression level of the exogenous gene in the host. The CAI of genes of prokaryotic origin is lower than that of the host core genes, but becomes closer and closer to a CAI value of the host core genes over time. The CAI of genes of eukaryotic origin has always been very close to that of the host core genes.

Fop refers to a ratio of optimal codons to their synonymous codons; the higher the Fop, the higher the frequency with which the optimal codons are used. Over time, the Fop of genes of prokaryotic origin becomes higher and higher, and the Fop of genes of eukaryotic origin has always been very close to that of the host core genes.

Nc is a measure of a degree of codon bias in a gene and quantifies an extent to which a gene uses same or equal synonymous codons in each amino acid class. Nc is a best overall estimator of absolute synonymous codon usage bias and can be easily calculated from codon usage data alone. For each gene, a value of Nc ranges from 20 (extreme bias when only one codon is used for each amino acid) to 61 (when all codons are used uniformly). A higher Nc value indicates a weaker codon usage bias. Nc values of genes transferred from eukaryotes and prokaryotes into plants both show a downward trend over time and become closer and closer to the core genes.

### (4) Gene Functional Annotation and Enrichment

The present invention collects annotation information in 9 major databases, which are OG1157, eggNOG, PANTHER, SuperFamily, InterProScan, GO, KEGG, Pfam, and KO. The present invention predicts a function of a query protein sequence based on a sequence with a minimum e-value of the query protein sequence in BLAST alignment. In addition, the present invention further performs annotation analysis on a complete gene set of a species using an eggNOG-mapper tool (European Molecular Biology Laboratory; http://eggnog-mapper.embl.de).

What is claimed is:

1. A method for screening cross-species horizontal gene transfer (HGT), comprising:
Step 1: constructing a background database comprising known protein sequences covering phylogenetic lineages of prokaryotes and eukaryotes, and removing highly homologous and redundant sequences;
Step 2: performing BLAST alignment on any two protein sequences in the background database, calculating an orthologous group association network based on reciprocal best hits for each gene pair combination, and clustering the orthologous group association network by using an unsupervised Markov clustering algorithm to generate a database containing a plurality of orthologous groups;
Step 3: for each query protein sequence to be analyzed, searching for homologous sequences in the background database, performing strict sequence alignment and trimming, constructing a single-gene phylogenetic tree, and evaluating a support value of each branch of the phylogenetic tree through at least one of an ultrafast bootstrap test, an SH-like approximate likelihood ratio test, an approximate Bayes test, and a fast local bootstrap probability test;
Step 4: retrieving a nested topology of the query protein sequence and sequences that are distantly related in terms of evolutionary distance in the single-gene phylogenetic tree constructed centered on the query protein sequence; screening for a potential cross-species HGT by defining a nested position and setting a minimum support node threshold; and recording monophyletic best matching sequences of the query protein sequence in a recipient group and a donor group;
Step 5: evaluating confidence value of the potential cross-species HGT by calculating an alien index, an HGT score support index, an HGT branch length support index, and a consensus hit support index thereof;
Step 6: estimating a time of occurrence of a cross-species horizontal transfer event by tracing the minimum taxonomic boundary of descendants of the cross-species HGT and the minimum taxonomic boundary of descendants of the donor; and providing a temporal context for the cross-species HGT in combination with molecular clock dating and archaeological or fossil evidence.

2. The method for screening cross-species HGT according to claim 1, wherein in step 1, data of the background database comprise all available protein sequences of reference sequences in NCBI and protein sequences from a marine microbial eukaryote transcriptome sequencing project.

3. The method for screening cross-species HGT according to claim 1, wherein in step 2, multiple sequence alignment and protein domain analysis are performed on sequences in each of the orthologous groups.

4. The method for screening cross-species HGT according to claim 1, wherein step 5 further comprises analyzing the potential cross-species HGT using homology alignment characteristics of genes on both upper and lower sides of an HGT gene locus, specifically comprising:
if the potential cross-species HGT is located in a chromosomal fragment and 50% of genes in the chromosomal fragment have best matches from other biological kingdoms, then removing the candidate genes of the potential cross-species HGT; or,
if the potential cross-species HGT is located in a chromosomal fragment and 50% of genes in the chromosomal fragment are identified as HGT genes, then removing the candidate genes of the potential cross-species HGT; or,
if at least one of three nearest upstream or downstream genes of the potential cross-species HGT has a best hit in another biological kingdom, then excluding the potential cross-species HGT; or,
when two or more potential cross-species HGTs are physically tightly linked and belong to a same gene family, the two or more potential cross-species HGTs are regarded as a single cross-species HGT event, and all these adjacent potential cross-species HGTs are retained.
